(19) United States
(12) Patent Application Publication
(10) Pub. No.: US 2025/0258292 A1
(43) Pub. Date: Aug. 14, 2025

(54) TILTED TRANSMITTER WITH ADDITIONAL K-SPACE COVERAGE

(71) Applicant: QT IMAGING, INC., Novato, CA (US)

(72) Inventors: Nasser Charles Pirshafiey, Trabuco Canyon, CA (US); David T. Borup, Salt Lake City, UT (US); James W. Wiskin, Novato, CA (US)

(21) Appl. No.: 19/051,161

(22) Filed: Feb. 11, 2025

Related U.S. Application Data
(60) Provisional application No. 63/552,188, filed on Feb. 11, 2024.

(51) Int. Cl. G01S 15/89; G10K 11/30; G10K 11/35

(52) U.S. Cl. CPC G01S 15/8993; G01S 15/8936; G01S 15/8995; G10K 11/30; G10K 11/35

(57) ABSTRACT

An imaging system is provided that includes: a transmitter array; and a receiver array, wherein a transmission signal is transmitted from the transmitter array at an angle with respect to the receiver array, wherein the transmission signal is structured to focus at a region within 10-50 percent of a distance to the axis of rotation.

### CROSS-REFERENCE TO RELATED APPLICATION(S)

This application claims the benefit of U.S. Provisional Application Ser. No. 63/552,188, filed Feb. 11, 2024.

### BACKGROUND

Imaging systems such as ultrasound imaging systems can include a transmitter and receiver for conducting transmission ultrasound. The transmitter and receiver can be configured to rotate around an axis of rotation so as to image an object such as a breast. It can be challenging to obtain clear images of a breast at a chest wall.

### BRIEF SUMMARY

Systems and methods for generating additional k-space coverage for imaging are provided.

In some aspects, the techniques described herein relate to an imaging system, including: a transmitter array; and a receiver array, wherein a transmission signal is transmitted from the transmitter array at an angle with respect to the receiver array, wherein the transmission signal is structured to focus at a region within 10-50 percent of a distance to the axis of rotation. In some cases, the focus is between 25-35% of the distance to the axis of rotation.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

### BRIEF DESCRIPTION OF THE DRAWINGS

FIG. 1 shows a tilted transmitter in an imaging system as described herein.

FIG. 2 shows a comparison for tilted transmitter results between a non-tilted transmitter and a tilted transmitter used to image a breast.

FIG. 3 shows a comparison for tilted transmitter results between a non-tilted transmitter and a tilted transmitter used to image a breast.

FIG. 4 shows a simulation phantom created in a computer simulation software.

FIG. 5A show results with a standard 60 mm focus.

FIG. 5B shows the results with a 30 mm close focus.

FIG. 6 shows an image plane close to the chest wall.

FIG. 7A shows an image using a 32 mm tall receiver (16 rows 2 mm pitch).

FIG. 7B shows a larger version of the images of FIG. 7A in axial view.

FIG. 8 shows a close comparison with two views.

FIG. 9A shows a close up of an image, indicating a cross-section line.

FIG. 9B shows a plot of quantitative values along the cross-section line for a comparison of the standard and close focus.

FIG. 10 shows images reflecting the effect of 1 mm vs 2 mm standard pitch elements.

FIG. 11 shows images using a 30 mm focus with 8 rows and 1 mm pitch.

FIG. 12 shows a plot of quantitative values as one moves vertically.

FIG. 13 shows a comparison of 16 element arrays at 60 mm focus and 30 mm focus.

FIG. 14 shows a plot comparing the 16 element 1 mm array with other configurations.

FIG. 15A and FIG. 15B illustrate how the combination of tilt and closeness are able to result in better images.

FIG. 16 shows a basic illustration of a plane wave.

FIG. 17 shows a diagram for an example described herein.

FIG. 18 shows a diagram for an example described herein.

### DETAILED DESCRIPTION

Systems and methods for generating additional k-space coverage for imaging are provided.

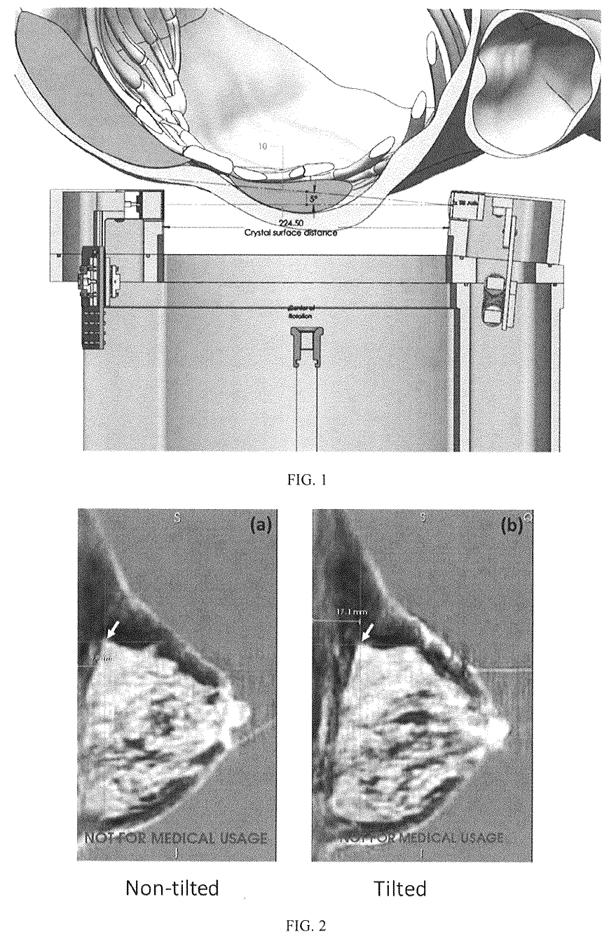

FIG. 1 shows a tilted transmitter. Referring to FIG. 1, by using a tilted transmitter, signal can penetrate the chest wall at an upward angle and bounces/scatters/reflects on the receiver. A 5 degree tilt is shown as an example. Other tilts with respect to each other and/or an axis of rotation are contemplated.

A tilted transmitter provides vertical visibility, which can provide improvements such as being able to acquire more signal closer to the chest wall, better image quality close to the chest wall from additional signal, and allow acquiring data above the water line (water being the acoustic coupling).

Certain embodiments described herein include, a tilted transmitter (Tx), tilted receiver (Rx), and a tilted Rx and Tx.

The tilted transmitter can be used to increase the k-space.

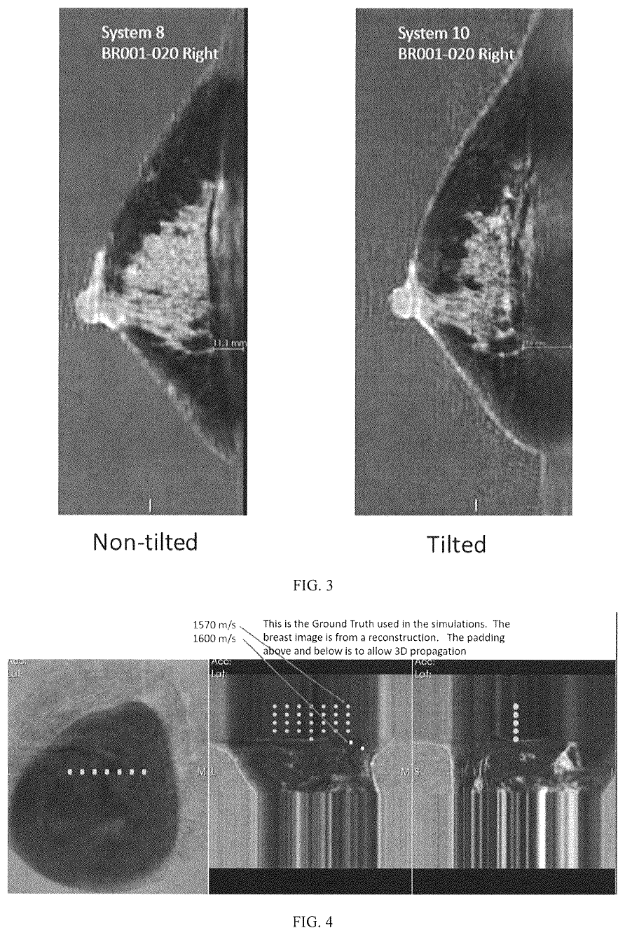

Example titled transmitter results are shown in FIG. 2 and FIG. 3, which show comparisons between a non-tilted transmitter and a tilted transmitter used to image a breast.

The k-space can be increased using the titled array and/or a close focus and beam steering capabilities. Beam steering is useful for improving SNR.

A close focus refers to the ability to focus the transmission beam within a region that is less than about 50 percent of a distance to the axis of rotation (or other central axis between a transmitter and receiver). Embodiments contemplate a close focus of between 20% and 50% of the distance to the axis of rotation.

The focus could also be dynamic. In some cases, the focus can be changed depending on the region of the breast being imaged. For example, the focus can be changed as one moves up the breast.

The mechanism to achieve the dynamic focus can be electronic or mechanical (or even material base). The tilt can also be dynamic with electronic or mechanical mechanisms.

For example, beam steering of the transmitter is possible provided the array pitch is less than λ/2. This flexibility in the tilt can be used both vertically and horizontally such that it is possible to dynamically steer the transmitted signal in all directions and in particular up or down and dynamically changing the focal distance.

The beam steering is 3D and allows for movement in pitch, yaw and roll. In particular, the dynamic changing of beam direction and or beam focus simultaneously or in conjunction with movement either around the breast or up and down may yield substantial improvement in image quality due to the increased coverage of k-space or the increased signal to noise ratio (SNR).

Other means for effectuating the focus include lens or other material in front of the transmitter, as well as using a curved transmitter.

It is also possible to perform dynamic shading of the intensity profile. The dynamic changing of the amplitude characteristics in the horizontal or vertical direction may aid in SNR and image quality. This dynamic change can take place as the arrays move up or down or around the breast and can be determined dynamically or with a preset program that is based on an initial scan of the breast or object being scanned.

Thus in certain embodiments an imaging system is provided that includes: a transmitter array; and a receiver array, wherein a transmission signal is transmitted from the transmitter array at an angle with respect to the receiver array, wherein the transmission signal is structured to focus at a region within 10-50 percent of a distance to the axis of rotation. In some cases, the focus is between 25-35% of the distance to the axis of rotation.

In some cases, the imaging system is structured to provide the focus as a fixed focus. For example, a lens component can be disposed in a path of the transmission signal, the lens component providing the fixed focus.

In some cases, the imaging system provides a dynamic focus. In some cases, the transmitter array includes a time-delay based focus control. In some cases, the dynamic focus is provided mechanically. In some cases, the transmitter array includes a movable focusing lens component. In some cases, the dynamic focus is provided by a lens having a controllable refractive index. In some cases, dynamic focus is provided electronically. In some cases, the dynamic focus is provided by a beam steering or beam forming technique. In some cases, the dynamic focus is provided by a 2D array of the transmitter array.

In some cases, the transmitter array is directed at an angle to the axis of rotation. In some cases, the receiver array is directed at an angle to the axis of rotation. In some cases, both the receiver array and the transmitter array are directed at angles to the axis of rotation.

In some cases, a method (which may be carried out by a controller) is provided that includes performing dynamic beam steering and dynamically changing the focal distance during an imaging operation using an imaging system such as described herein.

### Close Focus Experimental (Simulation) Validation.

Hypothesis: there is better k-space coverage with the close focus transmitter.

Validation: FIG. 4 shows a simulation phantom created in a computer simulation software. Synthetic forward data was generated using different vertical foci in vertical direction to emulate the scanner. Both 60 mm (standard) or 30 mm (close) focus was used. A different number of receivers (8 row vs 16 row receiver) were used. Full reconstruction—not a Born approximation was performed to generate the images.

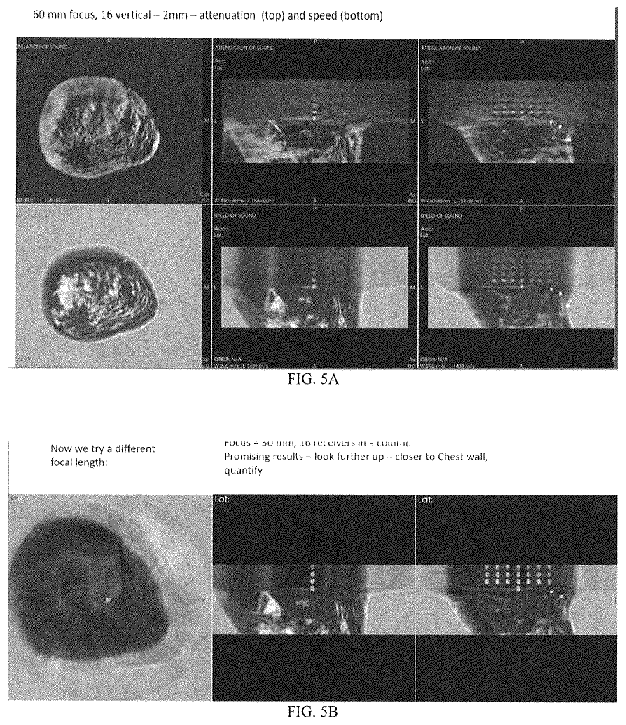

Results: The results with a standard 60 mm focus, including with a 16 row receiver array are shown in FIG. 5A. FIG. 5B shows the results with the 30 mm close focus. As can be seen, with the close focus, the reconstruction appears to be substantially better above the present imaging plane.

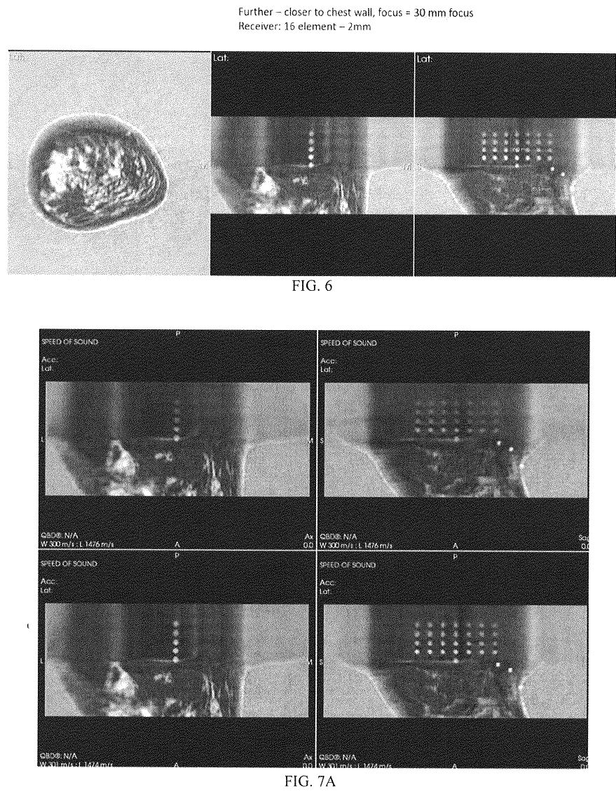

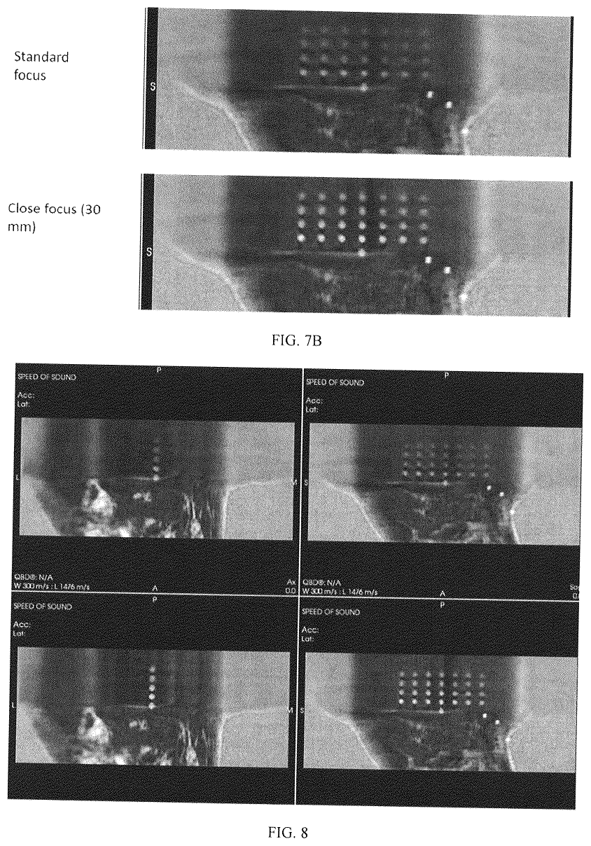

FIG. 6 shows an image plane close to the chest wall. Referring to FIG. 6, the image shows the reconstructed breast (simulation data) with measurements measuring height above the image plane. Substantial improvement was found 36 mm above the present image plane. FIG. 7A shows an image using a 32 mm tall receiver (16 rows 2 mm pitch). Referring to FIG. 7A, it can be seen that the 30 mm close focus performs much better higher up to the chest wall. In particular, the upper two images show a 2 mm 60 mm focus reconstruction and the bottom two images show a 2 mm 30 mm focus reconstruction. The tumors in the breast itself are more clear in the 30 mm focus images. FIG. 7B shows a larger version of the images of FIG. 7A in axial view. The improvement with close focus is more clearly seen. FIG. 8 shows a close comparison with two views.

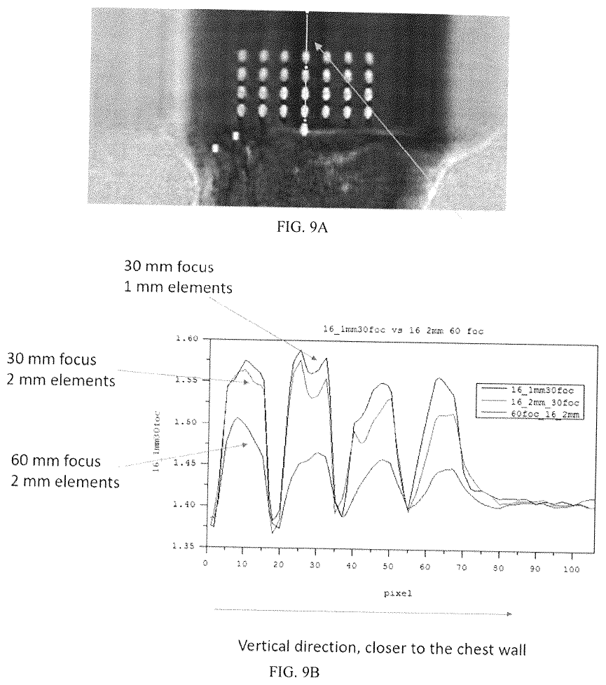

FIG. 9A shows a close up of an image, indicating a cross-section line. FIG. 9B shows a plot of quantitative values along the cross-section line for a comparison of the standard and close focus. Referring to FIG. 9B, it can be seen that even with the 16 row array, the 30 mm focus performs better than the 60 mm focus. The 1 mm elements perform better than the 2 mm pitch elements.

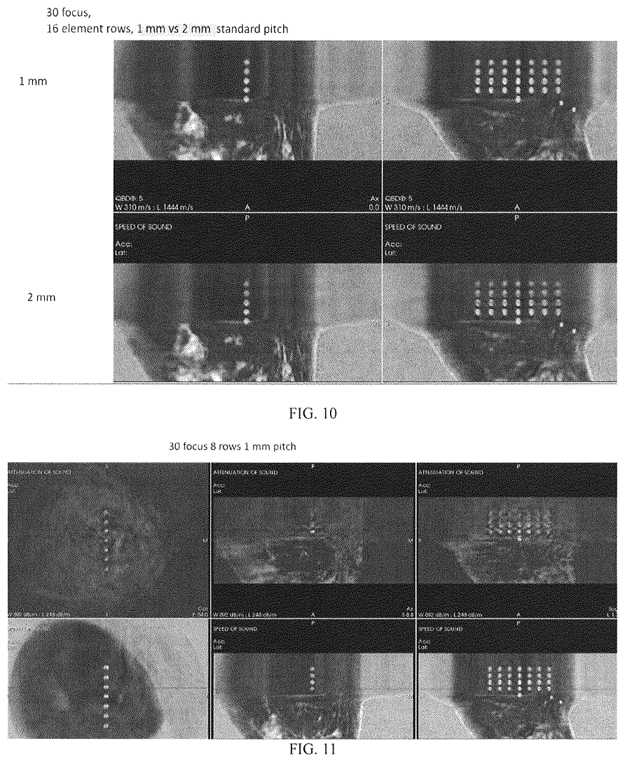

FIG. 10 shows images reflecting the effect of 1 mm vs 2 mm standard pitch elements. As can be seen, the 1 mm elements appear to yield a better reconstruction process.

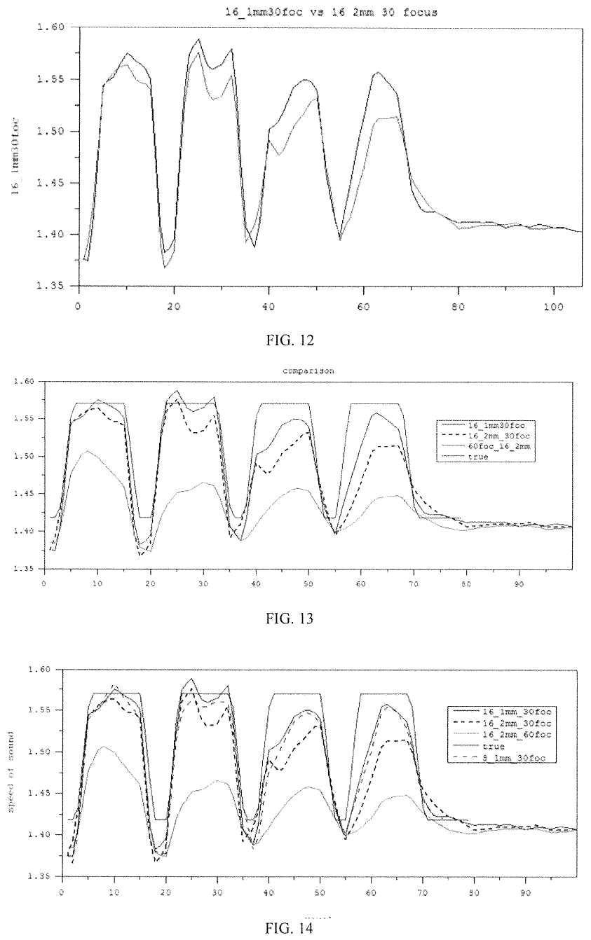

FIG. 11 shows images using a 30 mm focus with 8 rows and 1 mm pitch. Again, the 8 row receiver with 1 mm pitch appears to do well, even though it is only ½ the height of a 16 row receiver. Picking a vertical line, FIG. 12 shows a plot of quantitative values as one moves vertically. As can be seen, all things being equal, the 1 mm performs better than the 2 mm elements (16 elements vertically, each 2 mm tall). The x axis is distance from bottom.

FIG. 13 shows a comparison of 16 element arrays at 60 mm focus and 30 mm focus. Referring to FIG. 13, the 30 mm focus with 1 mm or 2 mm elements outperforms the 60 mm focus with 2 mm. The 1 mm elements perform better than 2 mm, but the 30 mm focus is superior for both cases. Note that you get closer to the chest wall as you move right. The x axis is the distance from the bottom of the image plane.

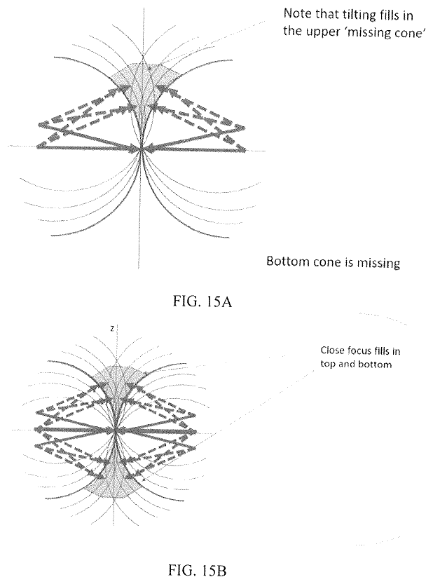

FIG. 14 shows a plot comparing the 16 element 1 mm array with other configurations. As can be seen in FIG. 14, the common element to the best performance is the 30 mm focus and 1 mm array pitch vertically. The quantitative performance of the 30 mm focus is almost independent of 16 vs 8 receivers. Clearly the 30 mm focus cases are substantially better than the 60 mm focus. This improved performance is clearly seen to be correlated highly with the 30 mm focus. 60 mm focus (lower line) performs universally worse than other configurations. There is little difference between 16 vs 8 row receivers as long as they have 30 mm focus.

FIG. 15A and FIG. 15B illustrate how the combination of tilt and closeness are able to result in better images. That is, additional coverage of k-space is possible through providing a tilt angle between the transmitter and receiver in transmission ultrasound and this additional coverage of the k-space can be leveraged by using a close focus.

In detail, with respect to FIG. 15A, we see that the 30 mm focus supplies substantially better images than the 60 mm focus. This is related to the success of the tilt. Note that tilting causes additional coverage in the k-space as per the diagram below. The green/shaded region is filled when the tilt is applied to the transmitter. The missing cone problem is an ongoing issue in tomography even with 3D ultrasound tomography (also called volography). Note however that substantially more region is filled in with the close focus approach. This is due to the spreading of the incident field after the focal region. Note also that these regions that are filled in become volumes when the 3D tomography is used and a second horizontal dimension is added. Imagine the image above rotating about the z-axis to see the effect of the 3D tomography. Note also that the resolution in the vertical direction is determined by these high k-wave number values that are filled in by the close focus. Note that this analysis is indeed a linear approximation to the full problem (it is based on the Born approximation to the full wave equation solution.

### Vertical Born Wave Space Components

Born approximation can be used to show that tilting the array in the vertical direction will result in additional space being filled in on the k-space region. Furthermore, the argument can be extended to show that there should be some advantage to having k-vectors in both the up and down directions. This can be approximately attained by the use of a close focus of 30 mm, The resulting diverging wave has k-wave vectors above and below the plan wave pointed directly at the receiver.

The derivation is immediate once the plane wave decomposition of the Green's function is used, and a plane wave is used as the incident field in the Born approximation. In fact, then everything is a Fourier Transform (or inverse) except for the square root factor, and the result follows directly. The basic idea can be best explained in terms of the Lippmann-Schwinger equation and the Born approximation in the special case of a plane wave travelling in the y direction:

$${u}^{s c} = {\int }_{Ω} {dr}^{′} G ( r - {r}^{′} ) γ ( {r}^{′} ) u ( {r}^{′} ) \to ( Born approximation ) \to {u}^{s c} \approx {\int }_{Ω} {dr}^{′} G ( r - {r}^{′} ) γ ( {r}^{′} ) {u}^{i n c} ( {r}^{′} )$$

Using a plane wave incident field with a vertical component for simplicity: u^{inc}(r′)=e^{ik}^{₀}^{y′+ik}^{_{z0}}^{z′}=e^{i(k}^{₀}^{,k}^{_{z0}}^{)(y′,z′)}, gives immediately: u^{sc}=∫_{Ω}dr′G(r−r′)γ(r′)e^{ik}^{_{y0}}^{y′}e^{ik}^{_{z0}}^{z′}, |k_{z0}|<<k_{y0}

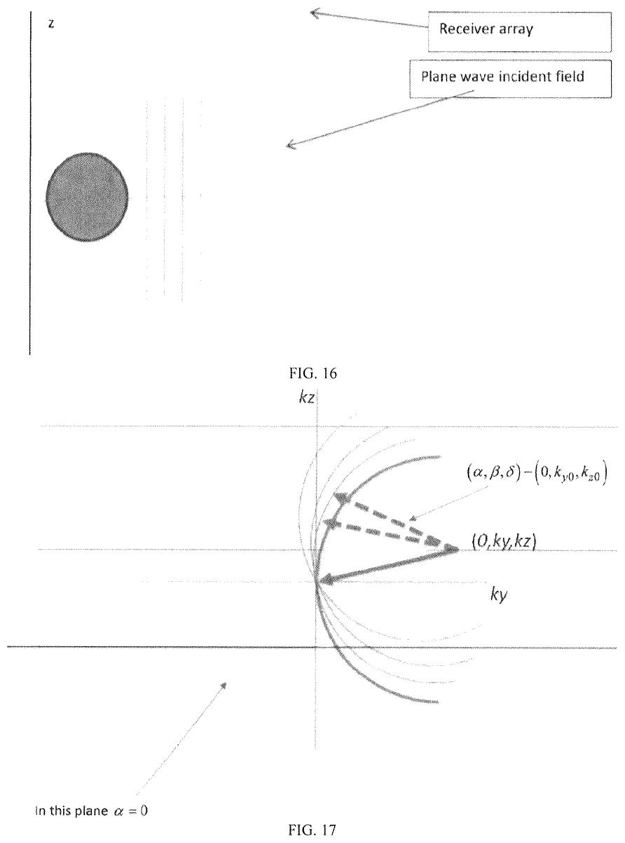

The stipulation |k_{z0}|<<k_{y0} allows us to keep the assumption about the plane wave: y₀−y′≥0, where y=y₀ is the position of the receiver array, as illustrated in FIG. 16, which shows a basic illustration of a plane wave.

The standard Weyl decomposition of the Green's function into plane waves gives:

$$G ( r - {r}^{′} ) = \frac{1}{π} \int d α d δ \frac{{e}^{i α ( x - {x}^{′} ) + i β \vert y - {y}^{′} \vert + i δ ( z - {z}^{′} )}}{β} ,$$

where β≡\sqrt{k₀²−α²−δ²} gives immediately

$${u}^{sc} ( x , {y}_{o} , z ) \approx \frac{1}{π} {\int }_{R} d α d δ \frac{{e}^{i β {y}_{0}}}{β} {e}^{i α x} {e}^{i δ z} {\int }_{Ω} {dr}^{′} {e}^{- {ix}^{′} α - {iz}^{′} ( δ - {k}_{z 0} ) - {iy}^{′} ( β - {k}_{y 0} )} γ ( {r}^{′} )$$

Which is:

$${u}^{sc} ( x , {y}_{o} , z ) \approx 4 π \frac{{e}^{i β {y}_{0}}}{4 πβ} {\int }_{R} d α d δ {e}^{i α x} {e}^{i δ z} {\int }_{Ω} {dr}^{′} {e}^{- {ix}^{′} α - {iz}^{′} ( δ - {k}_{z 0} ) - {iy}^{′} ( β - {k}_{y 0} )} γ ( {r}^{′} )$$

Clearly this has the form of a 3D Fourier Transform followed by inverse 2D Fourier Transform, except for the

$$\frac{{e}^{i β {y}_{0}}}{4 πβ} ≡ \frac{{e}^{i \sqrt{{k}_{0}^{2} - {δ}^{2} - {α}^{2}} {y}_{0}}}{4 π \sqrt{{k}_{0}^{2} - {δ}^{2} - {α}^{2}}} .$$

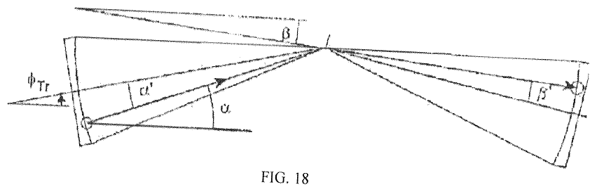

Thus,

$${\hat{u}}^{sc} ( {y}_{0} , α , δ ) ≡ F [ {u}^{sc} ( x ) ] \approx 4 π \frac{{e}^{i β {y}_{0}}}{\sqrt{{k}_{0}^{2} - {δ}^{2} - {α}^{2}}} F [ γ ] ( α , β - {k}_{y 0} , δ - {k}_{z 0} )$$

where F is the Fourier Transform. This is a specialized geometry, but by symmetry this is true irrespective of the angle of rotation of the transmitter/receiver pair. Note that the position in k-space is given by the difference of the two vectors (α,β,δ)−(0,k_{y0},k_{z0}) and that α²+β²+δ²=k₀²=k_{y0}²+k_{z0}². That is, both vectors are of length ∥(α,β,δ)∥=∥(0,k_{y0},k_{z0})∥=k₀. Thus the diagram shown in FIG. 17 applies. Referring to FIG. 17, the diagram shows what happens when the transmitter is tilted with respect to the receiver in the vertical direction. Note that applying this to the other side fills in the upper part of k-space more effectively. Note also that the multiplicity of wave numbers fills in the k-space more effectively. Note also (with reference to FIG. 15A), the upper part of k-space would be void if the additional k-vectors with vertical component in the incident field relative to the receiver array. Note also that the angle of the incident field relative to the receiver array is critical. Note SNR questions are independent. As previously alluded to in FIG. 15A, the wedge shaped/cone region is not covered in k-space and leads to artifacts and poor resolution when the inverse Fourier transform (or iterative equivalent) is applied to yield the spatial image. Note: with reference to the diagram of FIG. 18, k-vectors that correspond to a negative kz yield an infilling of the k-space in the lower ‘wedge’.

Returning to FIG. 17, and with reference to FIG. 15A, k-space coverage is best when there are wave vectors with both upward and downward z-components.

Although in his paper, Devaney makes use of the “integral representation” of the Hankel function since this is the Green's function for 2D. In reality this is the Weyl or plane wave decomposition of the Green's function associated with the Helmholtz equation. It is incidental that it is the Hankel function in 2D. So in 3D, the formula is proved merely by taking the inverse 3D Fourier Transform of the Fourier transform of the spatial Green's function. In 3D the inverse Fourier transform is:

$${\int }_{- ∞}^{∞} ( \int \int \frac{{e}^{ik \cdot x}}{k \cdot k - {k}_{o}^{2}} {dk}_{x} {dk}_{z} ) {dk}_{y}$$

The k_{y} integral is carried out by Contour integration and use of Jordan's lemma. The singularity at k_{y}=\sqrt{kₒ²−kₓ²−k_{z}²} is moved to the first quadrant by an addition of some attenuation, and evaluation of the residue gives the Weyl (plane wave) decomposition mentioned above. Note this applies in 2D in the same manner.

Accordingly, it has been shown by theory and simulation that close focus can provide improvements to image quality, especially near the chest wall, which is important for breast cancer detection. 30 mm focus is better than 60 mm for getting close to the chest wall. Both close focus and tilted transmitter/receiver array will cover missing information in k-space. Close focus will cover 2× times as much information in k-space.

It should be noted that while the simulations were conducted with 30 mm being a “close” focus and 60 mm being a standard focus, these values are based on a distance to an axis of rotation being about 110 mm. Other distances closer or farther than the 30 mm (even for a 110 mm distance to the axis of rotation) are contemplated for the close focus distance.

Although the subject matter has been described in language specific to structural features and/or acts, it is to be understood that the subject matter defined in the appended claims is not necessarily limited to the specific features or acts described above. Rather, the specific features and acts described above are disclosed as examples of implementing the claims and other equivalent features and acts are intended to be within the scope of the claims.

What is claimed is:

1. An imaging system, comprising:
a transmitter array; and
a receiver array, wherein a transmission signal is transmitted from the transmitter array at an angle with respect to the receiver array, wherein the transmission signal is structured to focus at a region within 10-50 percent of a distance to the axis of rotation.

2. The imaging system of claim 1, the imaging system being structured to provide the focus as a fixed focus.

3. The imaging system of claim 2, further comprising a lens component in a path of the transmission signal, the lens component providing the fixed focus.

4. The imaging system of claim 1, wherein the focus is between 25-35% of the distance to the axis of rotation.

5. The imaging system of claim 1, the imaging system being structured to provide a dynamic focus.

6. The imaging system of claim 5, wherein the transmitter array comprises a time-delay based focus control.

7. The imaging system of claim 5, wherein the dynamic focus is provided mechanically.

8. The imaging system of claim 5, wherein the transmitter array comprises a movable focusing lens component.

9. The imaging system of claim 5, wherein the dynamic focus is provided by a lens having a controllable refractive index.

10. The imaging system of claim 5, wherein the dynamic focus is provided electronically.

11. The imaging system of claim 10, wherein the dynamic focus is provided by a beam steering or beam forming technique.

12. The imaging system of claim 10, wherein the dynamic focus is provided by a 2D array of the transmitter array.

13. The imaging system of claim 1, wherein the transmitter array is directed at an angle to the axis of rotation.

14. The imaging system of claim 1, wherein the receiver array is directed at an angle to the axis of rotation.

15. The imaging system of claim 1, wherein both the receiver array and the transmitter array are at angles with respect to the axis of rotation.
